(19) United States
(12) Patent Application Publication
(10) Pub. No.: US 2022/0362045 A1
(43) Pub. Date: Nov. 17, 2022

(54) ENDOSCOPIC GASTROINTESTINAL DEVICE FOR RESTRICTION AND REDUCING FOOD ABSORPTION WITH A POSITIONING SYSTEM IN THE STOMACH AND EXTENSION TO THE INTESTINE

(71) Applicant: Gabriel CAIRO NUNES, Araraquara (BR)

(72) Inventor: Gabriel CAIRO NUNES, Araraquara (BR)

(21) Appl. No.: 17/767,287

(22) PCT Filed: Oct. 22, 2020
(86) PCT No.: PCT/BR2020/050431

§ 371 Date: Apr. 7, 2022

(30) Foreign Application Priority Data
Oct. 25, 2019 (BR) BR10 2019 0225262

(51) Int. Cl. A61F 5/00; A61F 2/04

(52) U.S. Cl. CPC A61F 5/0076; A61F 2/04; A61F 5/0079

(57) ABSTRACT

For desorptive technique, inhibiting hunger and reducing the amount of food ingested, besides avoiding the contact of the food with part of the intestine, working for the weight reduction of the patient and associated diseases such as type 2 diabetes. For this, a gastrointestinal device (10) formed from an intragastric ring (1) made of malleable and expandable material, extends a tubular sleeve (3) with or without a stent. The gastrointestinal device (10) has its inlet valve (4) attached to a catheter (CA) for gastrointestinal implant, and is directed into the stomach (L) of the patient, where an endoscopic forceps (EP) or the stent directs the tubular sleeve (3) through the pyloric canal (P), into the duodenum (D), and unfolding until it occupies part of the intestine. Therefore, the intragastric ring (1) is positioned near the Pyloric canal (P), inside the stomach (E), and is then inflated and expanded by fluid—liquid, gas or air, assuming the shape of a balloon.

### TECHNICAL FIELD

The present specification refers to an application for a patent of invention for a new combination of a communicating intragastric ring with an intestinal sleeve, with or without a stent, coupled to the intragastric ring.

This gastrointestinal device is guided deflated through the stomach of the patient by a stomach endoscopy procedure. The intragastric ring is positioned above the pyloric orifice and, after insufflation by fluid-liquid air or gas, from its entry valve (by the catheter that leads it), assuming a spherical shape free of tips or corners, occupying part of the stomach and anchoring the tubular sleeve, unfolded in the duodenum and in part of the intestine. Unfolding and positioning the sleeve can be done by endoscopic forceps or by stent (if available).

With this structure, even with reduced dimensions, the intragastric ring does not migrate to the intestine due to the size it reaches when inflated and due to the retention caused by the anchoring of the intestinal sleeve, remaining stabilized without the need for clips or ties and being minimally exposed in the stomach.

Therefore, part of the food processed by the stomach (with its walls completely free), passes through the hollow intragastric ring and also has its absorption flow reduced as it passes through the intestine lined by the tubular intestinal sleeve.

Therefore, the gastrointestinal device, occupying part of the stomach, acts in a restrictive way, inhibiting hunger and reducing the amount of food ingested. In a desorptive technique, the sleeve avoids the contact of food with part of the intestine, reducing absorption, working for the weight reduction of the patient and fighting diseases associated with this malady such as type 2 diabetes.

### Comments on the Technique

As is known not only by medical circles but also by society in general, overweight and obesity are present in more than 50% of the world's population. In developing countries like Brazil, these rates reach more than 60% of the population. Overweight and obesity bring with them large amounts of associated diseases called comorbidities, among them diabetes, hypertension, sleep apnea, osteoarticular diseases, and hepatic steatosis, some types of cancer and many other diseases. The amount of extra weight is directly related to the increased global death rate. Most of the treatments to combat this scourge are fleeting, and as a result, bariatric surgery has been gaining ground.

However, there are now intragastric devices that are introduced into the patient's stomach by a trans-oral procedure through the oral channel and are then expanded by fluid through the action of a valve.

In this regard the patent document BR 11 2019 006079 0 can be cited, which has two images included as examples, such as FIG. 1 for the patent application to be described later. As can be seen from these images, the device has a tubular body (2) fitted with stents or meshes (202), surrounded by a balloon structure (3) and which has a end ring (4), from which a duodenojejunal bypass sleeve (101) extends. The tubular body (2) is introduced by endoscopic procedure into the stomach with the balloon (3) deflated, and the end ring (4) is positioned below the pylorus. After inflation, the balloon (3), by its parts (31) and (32) lines the stomach walls and its duodenojejunal bypass sleeve (101) is extended, as lining, through the intestine of the patient.

Another patent document, BR 11 2015 000384 2, is cited and also has two images included as an example as FIG. 2 for the application to be described hereafter. As can be seen from these two images, the device is made up of a ring (220) with a tie (224) connecting to a sleeve (202) with a corrugated edge (2810) of elastic wire, or with an edge wrapped by a stent (422). When the device, folded, is transported inside the gastrointestinal tract by the elastic corrugation (2810) or the stent (422) the sleeve (202) is attached below the Pylorus and extends inside the intestine, lining the duodenal wall, keeping the ring (220), connected by the tie (224), positioned in the stomach of the patient. As can be seen, the ring (220) is attached by means of welds (228) to the stomach wall of the patient.

The device mentioned above is part of a method called “Endobarrier”, whose images, extracted from Google are inserted, also by way of example, as FIG. 3 for the application to be described later. It is a method developed for non-surgical treatment in patients with type 2 diabetes and obesity, still in experimental condition. As you can see from these images, the device is a waterproof Polytetrafluoroethylene membrane body that extends through the duodenum and is carried endoscopically with the patient under anesthesia. Its edge is cut out in a wave shape and has clips with ties for fastening just below the Pyloric orifice. The “Endobarrier” device is carried through the stomach of the patient via the normal operating channel of a standard gastroscope, by probe, receiving the filling fluid through its cut edge, inflating the membrane for the start of treatment.

Therefore, the known devices (gastrointestinal implants), such as those exposed above, may bring discomfort to the gastrointestinal tract of the patient during treatment. There is a concern to avoid occlusion, migration of the intragastric element, and also other factors that can impair the treatment such as undesirable sealing (marked contact of an inflated balloon) with the stomach walls (FIG. 1), or needing anchor points, by means of staples, clips and ties, which can perforate the stomach or intestine walls (FIGS. 2 and 3).

### Objectives of the Invention

The insufflable gastrointestinal endoscopic device, the subject of this patent application, due to its constructiveness and the system that surrounds it, proposes the positioning of an intragastric ring immediately above the pyloric orifice, keeping it stabilized and providing the frame of an intestinal sleeve in the duodenum and part of the intestine, avoiding perforations and leaving the stomach walls (which naturally suffer contractions) totally free to process food.

For this, a valve is installed in the body of the intragastric ring, which extends a tubular sleeve, equipped or not with a stent (spiral or rectilinear). By the proposed positioning system, through its valve connected to a catheter, this intragastric ring assembly coupled with the intestinal sleeve is brought entirely folded to the Pylorus. After positioning the intragastric ring above the entrance to the pyloric canal, the ring is insufflated with air, gas or liquid, and the intestinal sleeve is then extended through the duodenum and part of the intestine with the aid of endoscopic forceps or the stent. In this condition, the intragastric ring, inflated like a balloon, is stabilized above the entrance to the pyloric canal without being attached and the intestinal sleeve remains distended along the walls of the duodenum and part of the intestine.

Therefore, without the use of any kind of staple, clip, tie or lining on the stomach walls, the intragastric ring stabilized in this way allows part of the processed food to pass through and this part of the processed food to pass through the intestinal sleeve along the duodenum and part of the intestine, thereby reducing the absorption of the ingested food content and reducing the absorption of the food by the intestine, resulting in a treatment against obesity and diseases associated with excess weight.

The gastrointestinal device remains so, acting as a restrictive and desorptive factor (such as a gastric bypass surgery), but without the risk of surgery, and it is also reversible or reapplicable.

### BRIEF DESCRIPTION OF THE FIGURES

After a superficial explanation, the invention is now detailed.

FIGS. 1, 2 and 3, as already explained, refer to the prior art, shown through patent documents BR 11 2019 006079 0, BR 11 2015 000384 2 and by the images of the “Endobarrier” device.

The following figures, from 4 to 14, show the intragastric ring coupled to the intestinal sleeve and its positioning system, reasons for this patent application, to be better detailed through the figures listed below:

FIG. 4—illustrates the gastrointestinal device with the corrugated tubular intestinal sleeve;

FIG. 5—illustrates the gastrointestinal device in a configuration where the tubular sleeve adopts a spiral type stent;

FIG. 6—illustrates the gastrointestinal device in a configuration where the tubular sleeve adopts a rectilinear type stent;

FIG. 7—illustrates the gastrointestinal device being directed into the stomach, aided by the catheter, in an endoscopy procedure;

FIG. 8—view of the intragastric ring of the device already positioned above the pyloric canal, with the intestinal sleeve being extended through the duodenum and part of the intestine with the help of endoscopic forceps;

FIG. 9—illustrates the positioned device, with the ring filling fluid (liquid, air or gas) being injected from the catheter and through the valve, when the intragastric ring is insufflated like a balloon until it takes the shape of the lower stomach walls. With this, the intragastric ring is kept stabilized above the pyloric channel and minimally exposed in the stomach;

FIG. 10 illustrates the above positioning, with the ring insufflated, where the catheter is removed and the device is ready to perform its functions;

FIG. 11—illustrates the positioning system shown in the previous figures, but with the tubular sleeve equipped with a stent, which is insufflated together with the ring, unfolding the tubular sleeve through the duodenum and part of the intestine without the need for endoscopic forceps;

FIG. 12—illustrates the device positioned on the patient, who, when feeding, has the processed food directed by the central nozzle of the inflated ring and directed by the sleeve, where the device acts in a restrictive and desorptive way;

FIGS. 13 and 14—show a photo and a detail of the device being held by the inventor, at a top and a front angle, respectively.

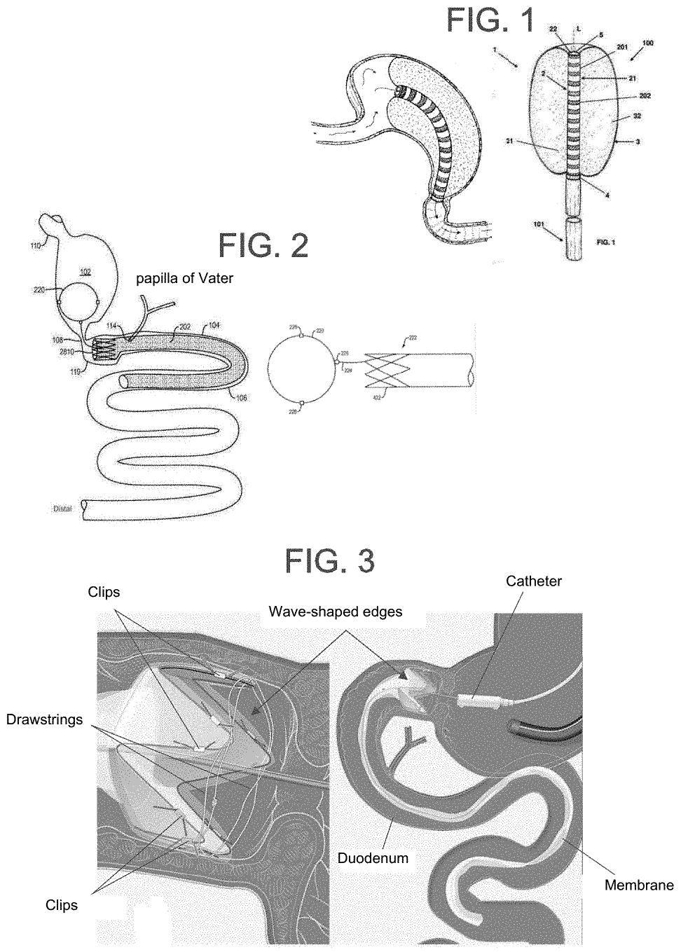

### DETAILED DESCRIPTION OF THE INVENTION

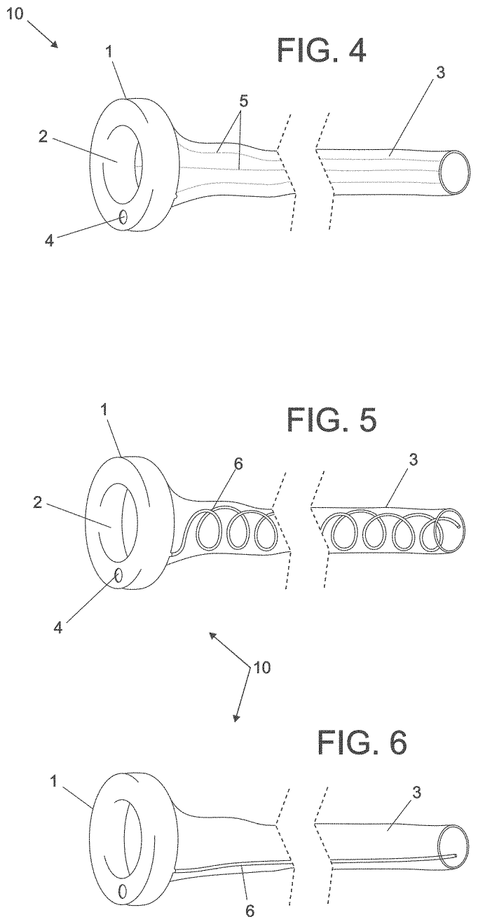

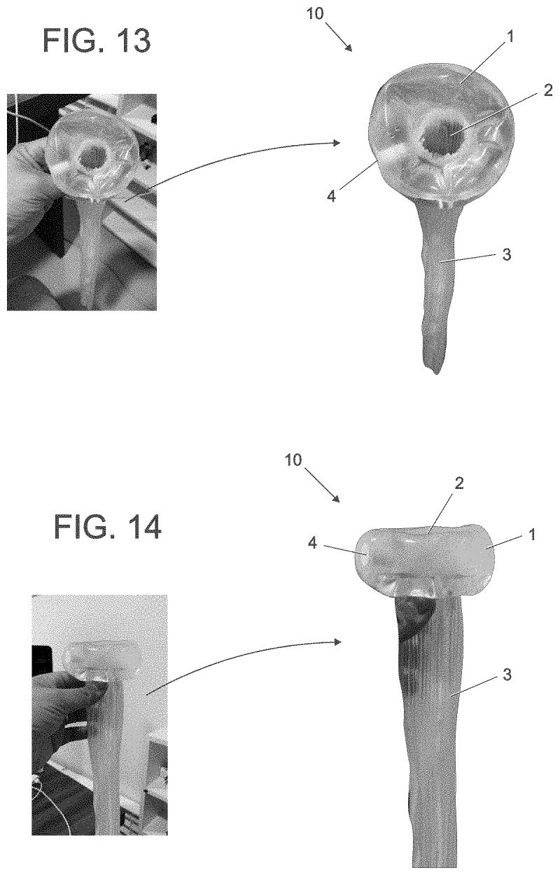

According to the attached figures, the “ ENDOSCOPIC GASTROINTESTINAL DEVICE FOR RESTRICTION AND REDUCING FOOD ABSORPTION WITH A POSITIONING SYSTEM IN THE STOMACH AND EXTENSION TO THE INTESTINE”, object of this invention patent application, as illustrated in FIGS. 4, 13 and 14, a gastrointestinal device (10) formed from an intragastric ring (1) produced in malleable and expandable material, preferably silicone, where from its central portion (2) extends a tubular sleeve (3), made of the same material.

Said device designed to have its intragastric ring (1) positioned and inflated in the stomach of a patient and extend its sleeve (3) to the intestine in order to reduce food absorption.

More specifically, the intragastric ring (1) is in a retracted, deflated, and compact state. The ring (1) adopts, at any place of its expandable body, an inlet valve (4), of the type with outlet lock and whose nozzle is suitable to receive the tip of a catheter (CA).

More specifically, the tubular sleeve (3) is welded and permanently connected to the ring (1), and has its body corrugated into bending ribs (5) to allow for expansion and retraction. The end of the sleeve (3) connected to the ring (1) coincides with its central opening (2), from where it extends its tubular body to the opposite end, the length of which can vary depending on the application or patient anatomy.

The tubular sleeve (3) can be equipped with a hollow stent (6) of the spiral type, as illustrated in FIG. 5, or of the rectilinear type, as illustrated in FIG. 6. This stent (6) has its length extended along the length of the tubular sleeve (3) and can have its air, liquid or gas inlet end connected directly to the intragastric ring (1) or not, where it then receives an independent valve with catheter inlet.

Once the gastrointestinal device (10) is formed, it is configured to reside in the stomach and intestine of the patient and resist migration, to effect both a weight reduction treatment and to combat diseases associated with this malady, such as type 2 diabetes.

For its positioning system, the gastrointestinal device (10) has its inlet valve (4) attached to a catheter (CA) for gastrointestinal implant, i.e. for introduction into the stomach (E) of a patient. When not equipped with a stent (6), an endoscopic forceps (EP) is inserted into the central opening (2) of the ring (1), resting on the sleeve (3). Therefore, the gastrointestinal device (10) is directed into the stomach of the patient. The reduced thickness of both the sleeve (3) and the intragastric ring (1) allow for introduction into the stomach (E) with minimal discomfort for the patient.

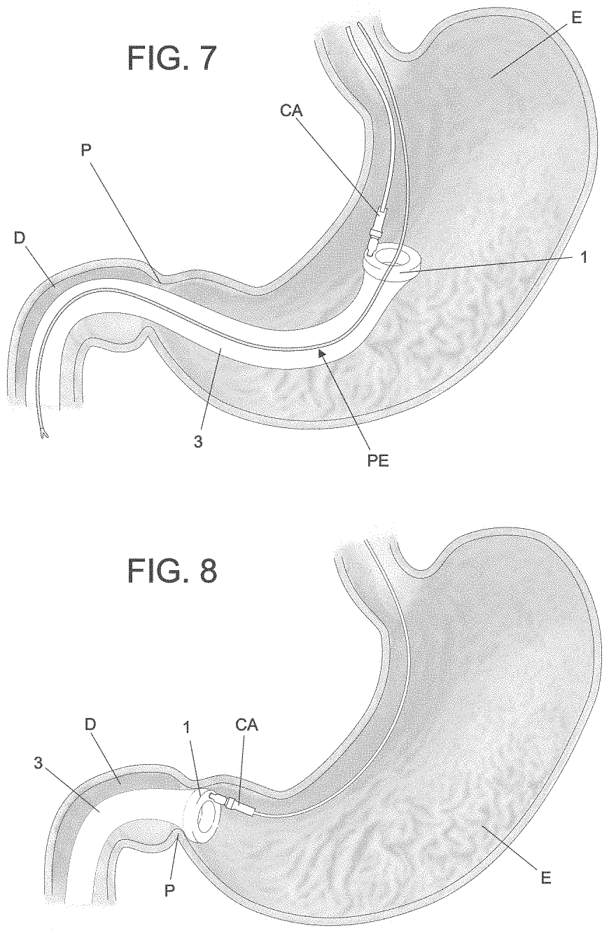

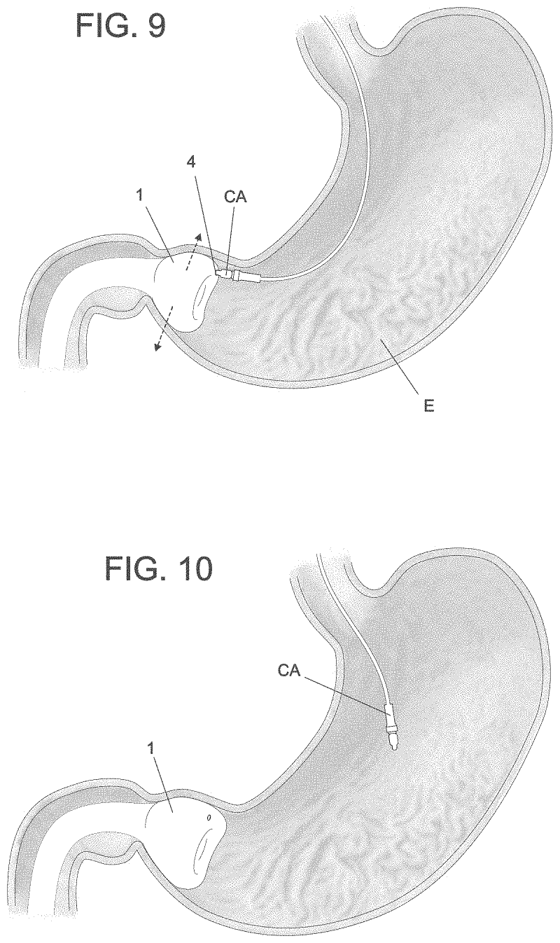

As illustrated in FIG. 7, the endoscopic forceps (EP) guides the tubular sleeve (3) through the pyloric canal (P), entering the duodenum (D) and unfolding until it occupies part of the intestine. The intragastric ring (1), deflated, is then positioned next to the pyloric canal (P), inside the stomach (E). Therefore, when the release of fluid (liquid, air or gas) is triggered by the catheter (CA), it enters from the inlet valve (4), as illustrated in FIGS. 9 and 10, inflating and expanding the intragastric ring (1) above the pyloric channel (P), thus configuring itself as a balloon of spherical shape or close to it and free of tips or corners. The intra gastric ring (1) then remains in this position, without the possibility of being moved, and the catheter (OC) and endoscopic forceps (EP) can then be removed.

The positioning of the tubular sleeve (3) in the duodenum and intestine can be performed before or after insufflation of the intragastric ring (1).

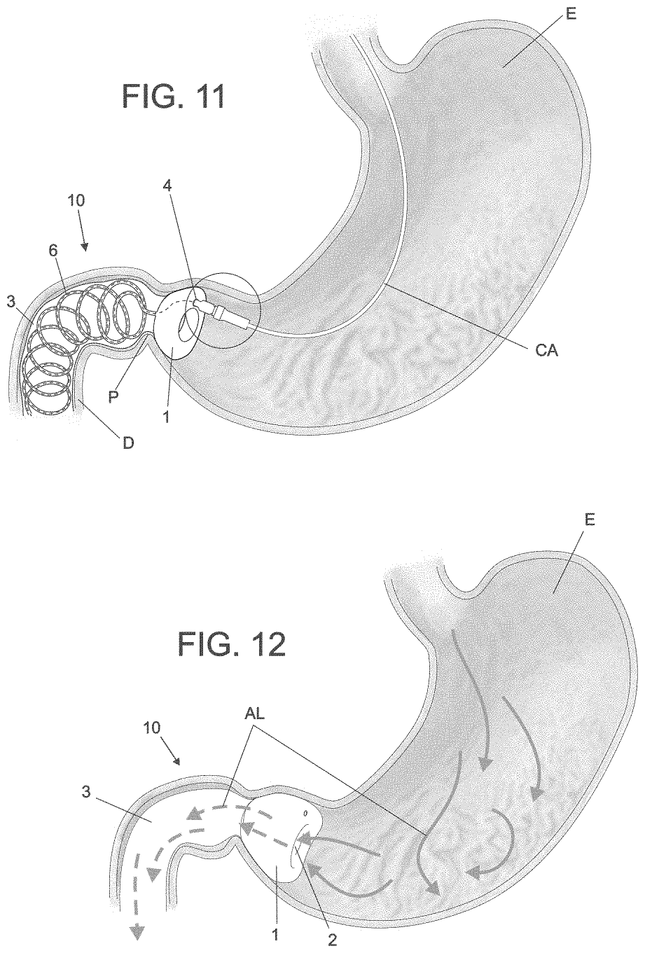

Alternatively, as illustrated in FIG. 11, when equipped with a stent (6) (spiral or rectilinear), the tubular sleeve (3) is only positioned at the entrance of the duodenum (D) when positioning the device (10) in the stomach (E). This way, if the stent (6) is connected directly to the valve (4) of the ring (1), it will also receive the fluid supply—liquid, air or gas—inflating and assembling its body, extending and positioning the tubular sleeve (3) in the duodenum and intestine, without the need for endoscopic forceps. If the stent (6) is separated from the ring (1), a second catheter is inserted into its valve before the device (10) is inserted into the stomach (E). Thus, when correctly positioned, before or after the ring (1) is inflated, it receives its own supply of fluid to arm its body and unfold the sleeve (3) through the duodenum and part of the intestine.

In either case, the inflated intragastric ring (1) remains stabilized just above the pyloric canal (P), without the need for the use of clips or ties like in common applications.

The intragastric ring (1) thus stabilized, as illustrated in FIG. 12, allows the passage of part of the processed food (AL) in restrictive technique by occupying part of the stomach, inhibiting hunger and reducing the amount of food ingested. The device (10) provides the passage of this part of processed food through the tubular sleeve (3), stabilized along the duodenum and part of the intestine, which results in a desorptive technique, preventing the absorption of the ingested food content in that part of the intestine. Therefore, by combining two techniques in a simplified construction and easy positioning without the need for surgery, the device (10) proves to be extremely effective in the treatment of obesity and diseases associated to excess weight.

The gastrointestinal device (10) remains positioned this way and stable, acting in a restrictive and desorptive way (such as a gastric bypass surgery), but without the risk of surgery, and it is also reversible or reapplicable.

1- “GASTROINTESTINAL ENDOSCOPIC DEVICE FOR FOOD RESTRICTION AND DESORPTION”, for gastrointestinal implant in the treatment of weight reduction and associated diseases such as type 2 diabetes by combining restrictive technique with desorptive technique, being a gastrointestinal device (10) formed from an intragastric ring (1) produced in malleable and expandable material, preferably silicone, characterized by, from its central hollow portion (2), extending a tubular sleeve (3) of the same material, the device being designed to have its intragastric ring (1) positioned and inflated in the stomach of a patient at the same time that it extends its sleeve (3) to the intestine in order to reduce food absorption.

2- “GASTROINTESTINAL ENDOSCOPIC DEVICE FOR FOOD RESTRICTION AND DESORPTION”, according to claim 1, characterized by the intragastric ring (1) being in a retracted, deflated and compact state, which adopts, anywhere in its expandable body, an inlet valve (4) of the type with outlet blockage and whose nozzle is suitable to receive the tip of a catheter (CA).

3- “GASTROINTESTINAL ENDOSCOPIC DEVICE FOR FOOD RESTRICTION AND DESORPTION”, according to claim 1, characterized by the tubular sleeve (3) being welded and permanently connected to the ring (1), having its body corrugated in fold ribs (5) to allow its expansion and retraction, with the end of the sleeve (3) connected to the ring (1) coinciding with its central opening (2), from where it extends its tubular body to the opposite end, in a variable length according to the application or anatomy of the patient.

4- “GASTROINTESTINAL ENDOSCOPIC DEVICE FOR FOOD RESTRICTION AND DESORPTION” according to claim 3, characterized by the tubular sleeve (3) being equipped with a hollow stent (6) of the spiral or rectilinear type.

5- “GASTROINTESTINAL ENDOSCOPIC DEVICE FOR FOOD RESTRICTION AND DESORPTION” according to claim 4, characterized by the stent (6) having its length extended along the length of the tubular sleeve (3) and having its air, liquid or gas inlet end connected directly to the intragastric ring (1) or being independent when it receives its own valve with catheter inlet.

6- “POSITIONING SYSTEM IN THE STOMACH AND EXTENSION TO THE INTESTINE”, according to the gastrointestinal device (10) defined in claim 1, which is configured to reside in the stomach and intestine of the patient and resist migration, without the need for clips or ties, inhibiting hunger, allowing the passage of only part of the processed food (AL) and preventing the passage of this part of processed food in part of the intestine, and for its positioning, the device (10) has its inlet valve (4) coupled to a catheter (CA) for gastrointestinal implant, characterized by, when not equipped with a stent (6), an endoscopic forceps (EP) is inserted into the central opening (2) of the ring (1), resting on the sleeve (3), and the gastrointestinal device (10) is then directed into the stomach (L) of the patient, where the endoscopic forceps (EP) directs the tubular sleeve (3) through the Pyloric canal (P), entering the duodenum (D) and unfolding until it occupies part of the intestine and the deflated intragastric ring (1) is positioned next to the Pyloric canal (P) inside the stomach (E); so, when the fluid (liquid, air or gas), air or gas is released through the catheter (CA), it enters from the inlet valve (4), inflating and expanding the intragastric ring (1) above the pyloric canal (P), configuring itself as a balloon that remains in this position, without being able to move, when then the catheter (CA) and the endoscopic forceps (EP) can be removed.

7- “POSITIONING SYSTEM IN THE STOMACH AND EXTENSION TO THE INTESTINE” according to claim 6, characterized by the positioning of the tubular sleeve (3) in the duodenum and intestine can be performed before or after the inflation of the intragastric ring (1).

8- “POSITIONING SYSTEM IN THE STOMACH AND EXTENSION TO THE INTESTINE”, according to claim 6, characterized by, when provided with a stent (6) (spiral or rectilinear), the tubular sleeve (3) is only positioned at the entrance of the duodenum (D) when positioning the device (10) in the stomach (E), where the stent (6) connected directly to the valve (4) of the ring (1) also receives the fluid supply—liquid, air or gas—directed to the latter, inflating and assembling its body, extending and positioning the tubular sleeve (3) in the duodenum and intestine, without the need for endoscopic forceps.

9- “POSITIONING SYSTEM IN THE STOMACH AND EXTENSION TO THE INTESTINE” according to claim 8, characterized in that the stent (6) separated from the ring (1) receives its own catheter, inserted into its valve before the device (10) is inserted into the stomach (E) to, when correctly positioned, before or after the ring (1) is insufflated, it receives its own supply of fluid to assemble its body and unfold the sleeve (3) through the duodenum and part of the intestine.
